(19) United States
(12) Plant Patent Application Publication
(10) Pub. No.: US 2023/0093795 P1
(43) Pub. Date: Mar. 23, 2023

(54) CANNABIS PLANT NAMED POI

(50) ;

(71) Applicant: Umami Worldwide, LLC, Las Vegas, NV (US)

(72) Inventor: Chase Martin, Las Vegas, NV (US)

(21) Appl. No.: 17/950,094

(22) Filed: Sep. 21, 2022

Related U.S. Application Data
(60) Provisional application No. 63/247,289, filed on Sep. 22, 2021.

(51) Int. Cl. A01H 6/28

(52) U.S. Cl. PLT/258

(57) ABSTRACT

The Cannabis cultivar POI can be briefly characterized by massive purple, white, green, and yellow streaking resin-coated flowers. With massive flower clusters and very short internodal space, this cultivar is able to produce very high yields for both flower and hash production.

Latin name of the genus and species: Cannabis sativa.

Variety denomination: ‘POI’.

### BACKGROUND OF THE INVENTION

The present invention relates to a new and distinct Cannabis cultivar designated “POI”.

### TAXONOMY AND NOMENCLATURE

Cannabis, more commonly known as marijuana, is a genus of flowering plants that includes at least three species, Cannabis sativa, Cannabis indica, and Cannabis ruderalis as determined by plant phenotypes and secondary metabolite profiles. In practice however, Cannabis nomenclature is often used incorrectly or interchangeably. Cannabis literature can be found referring to all Cannabis varieties as “sativas” or all cannabinoid producing plants as “indicas”. Indeed, the promiscuous crosses of indoor Cannabis breeding programs have made it difficult to distinguish varieties, with most Cannabis being sold in the United States having features of both sativa and indica species.

Human cultivation history of Cannabis dates back 8000 years (Schultes, R E., 1970, Random thoughts and queries on the botany of Cannabis. Pages 11-38 in: C R B Joyce, and S H Curry eds., THE BOTANY AND CHEMISTRY OF CANNABIS. J. & A. Churchill. London, England). Hemp cloth recovered in Europe dates back 6000 years (Small, E, Beckstead, H D, and Chan, A, 1975, The evolution of cannabinoid phenotypes in Cannabis, ECONOMIC BOTANY 29(3):219-232). The written record of the pharmacologic properties of Cannabis goes back more than 4000 years (Ti, H. 2737 BC. NEI JING SU WEN HUANG TI, Yellow Emperor's Classic on Internal Medicine; referred to without citation in Small et al. 1975 Supra).

The taxonomy and nomenclature of the highly variable genus Cannabis (Emboden, W A, 1974, ECONOMIC BOTANY 28(3):304-310; Small, E and Cronquist, A, 1976, TAXON 25(4):405-435; Small E and Cronquist, A, 1977, TAXON 26(1):110; Hillig, K W and Mahlberg, P G, 2004, American Journal of Botany 91(6):966-975), remains in question. This is in spite of the fact that its formal scientific name, ‘Cannabis sativa L.’, assigned by Carolus Linneaus (Linnaeus, C, 1753, SPECIES PLANTARUM, 2:1027, Salvius, Stockholm, Facsimile edition, 1957-1959, Ray Society, London, U.K.), is one of the oldest established names in botanical history and is still accepted to this day. Another species in the genus, ‘Cannabis indica Lam.’ was formally named somewhat later (de Lamarck, J B, 1785, ENCYCLOPEDIE METHODIQUE DE BOTANIQUE, 1(2):694-695), but is still very old in botanical history. In 1785, Jean-Baptiste Lamarck published a description of a second species of Cannabis, which he named Cannabis indica. Lamarck based his description of the newly named species on plant specimens collected in India. C. indica was described as relatively short, conical, and densely branched, whereas C. sativa was described as tall and laxly branched (Schultes R. E. et al, 1974, Harvard University Botanical Museum Leaflets, 23:337-367). C. indica plants were also described as having short, broad leaflets whereas those of C. sativa were characterized as relatively long and narrow (Anderson L. C., 1980, Harvard University Botanical Museum Leaflets, 28:61-69). C. indica plants conforming to Schultes' and Anderson's descriptions may have originated from the Hindu Kush mountain range. Because of the often harsh and variable (extremely cold winters, and warm summers) climate of those parts, C. indica is well-suited for cultivation in temperate climates.

Three other species names were proposed in the 1800s to distinguish plants with presumably different characteristics (C. macrosperma Stokes, C. chinensis Delile, C. gigantean Vilmorin), none of which are accepted today, although the epithet “Indica” lives on as a subspecies of C. sativa (‘C. sativa ssp. indica Lam.’, Small and Cronquist 1976 Supra).

In the 20th century, two new names were added to the 50 liturgy of proposed ‘Cannabis species: C. ruderalis’ Janischevsky and a hybrid, x ‘C. intersita’ Sojak. (Small, E, Jui, P Y, and Lefkovitch, L P, 1976, SYSTEMATIC BOTANY 1(1):67-84; Small and Cronquist 1976 Supra). Further, numerous names have been proposed for horticultural variants of ‘Cannabis’ but as of 1976, “very few of these have been validly published as formal taxa under the International Code of Botanical Nomenclature” (Small and Cronquist 1976 Supra). Moreover, other recent work continues to focus on higher-order evolutionary relationships of the genus. Cannabis has been variously ascribed as belonging to mulberry family (Moraceae) (Engler, H G A, Ulmaceae, Moraceae and Urticaceae, pages 59-118 in: A. Engler and K. Prantl eds., 1889, DIE NATURLICHEN PFLANZENFAMILIEN 3(1). W. Engelmann, Leipzig, Germany; Judd, W S, Sanders, R W, and Donoghue, M J, 1994, HARVARD PAPERS IN BOTANY 5:1-51; Humphries, C J and Blackmore, S, A review of the classification of the Moraceae, pages 267-277 In: Crane and Blackmore 1989 id.); nettle family (Urticaceae) (Berg, C C, Systematics and phylogeny of the Urticales, pages 193-220, in: P. R. Crane and S. Blackmore eds., 1989, EVOLUTION, SYSTEMATIC, AND FOSSIL HISTORY OF THE HAMAMELIDAE, VOL. 2, HIGHER HAMAMELIDAE, Clarendon Press, Oxford, U.K.); and most recently in its own family with hops (Humulus), Cannabaceae, or hemp family (Sytsma, K J, et al, 2002, AMERICAN JOURNAL OF BOTANY 89(9): 1531-1546). While the work of Small and Cronquist 1976 Supra, seemed to effectively confine the genus to a single species with 2 subspecies (C. sativa s., C. s. indica), each with two varieties (C. s. s. var. sativa, C. s. s. var. spontanea; C.s. i. var. indica, C. s. i. var. Kafiristanica) largely on the basis of chemotaxonomy and interfertility of all forms, more recent work (Sytsma et al. 2002 Supra), proposes a two-species concept, resurrecting the binomial C. indica Lam. Since Sytsma et al. (2002) provides no key for discriminating between the species, the dichotomous key of Small and Cronquist (1976), which accounts for all forms in nature, whether wild or domesticated, is preferred to classify the characteristics of the plants.

### BRIEF SUMMARY OF THE INVENTION

The present invention relates to a new and distinct Cannabis cultivar designated “POI”.

POI is an f1 hybrid cultivar. The mother is a poly hybrid mix of Purple Payne Killer, Durban Poison, Gelato 41, and Motorbreath ♯15. The mother plant is a rare outlying phenotype that can yield 90+ grams/ft2 under LED lighting and test at over 30% THC. The father is the proprietary f1 breeder stud Zuchi.

POI produces massive purple, white, green, and yellow streaking resin-coated flowers. With massive flower clusters and very short internodal space, this cultivar is able to produce very high yields for both flower and hash production. Select phenotypes have been able to yield 100+ grams of manicured flower /ft2 of canopy space.

It is a very hardy plant that is somewhat drought and bug resistant. Growing as much as 2+ inches per day with proper feed and environment.

The below numbers are the result of 1 selected phenotype from a population of only 10 seeds.

TOTAL CANNABINOIDS 31.78% THC

TOTAL TERPENES 2.564%

0.286% Alpha Pinene

0.189% Beta Pinene

0.235% Myrcene

0.106% Limonene

<0.002% Ocimene

<0.002% Terpinolene

0.187% Linalool 1.128%

Caryophyllene 0.433%

Humulene

### BRIEF DESCRIPTION OF THE DRAWING

FIG. 1 is a view of a POI leaf.

FIG. 2 is a view of several leaves and stems on a growing POI plant.

FIG. 3 is a view of leaves on a growing POI plant.

FIG. 4 is a view of a POI plant.

FIG. 5 is a view of a POI plant.

FIG. 6 is a view of young leaves and a meristem on a growing POI plant.

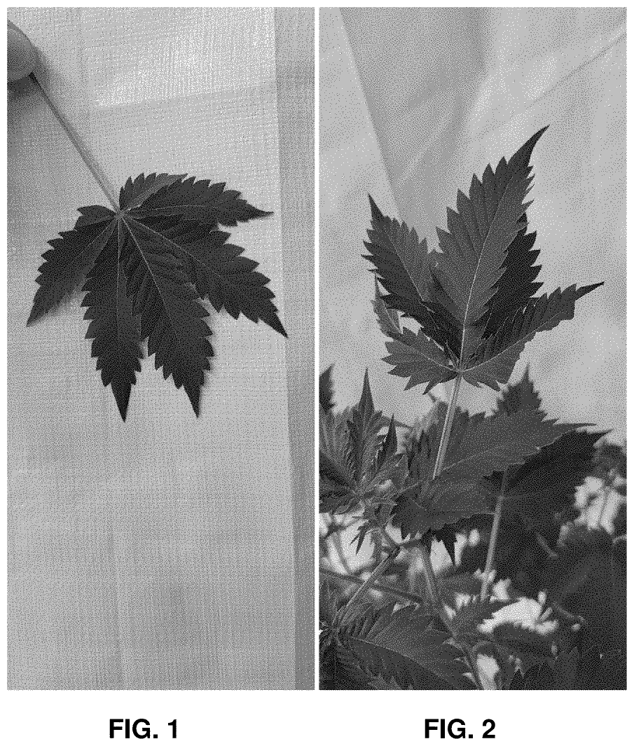

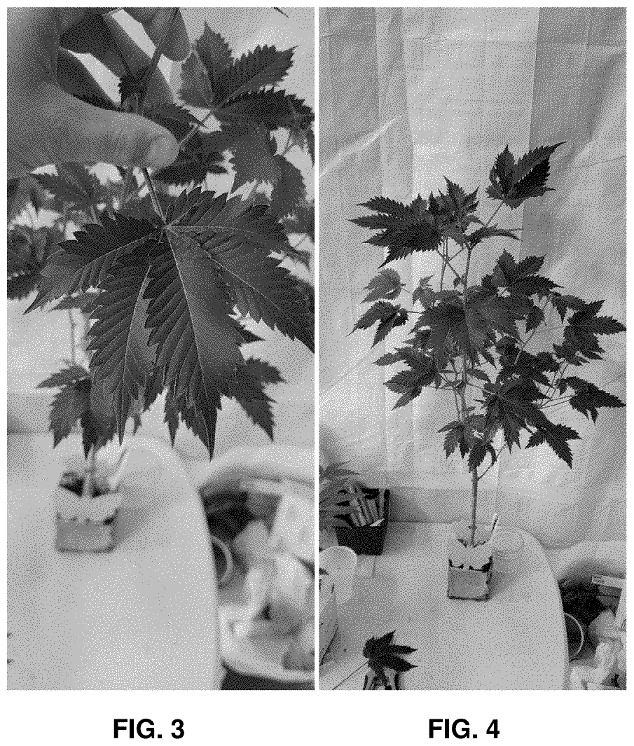

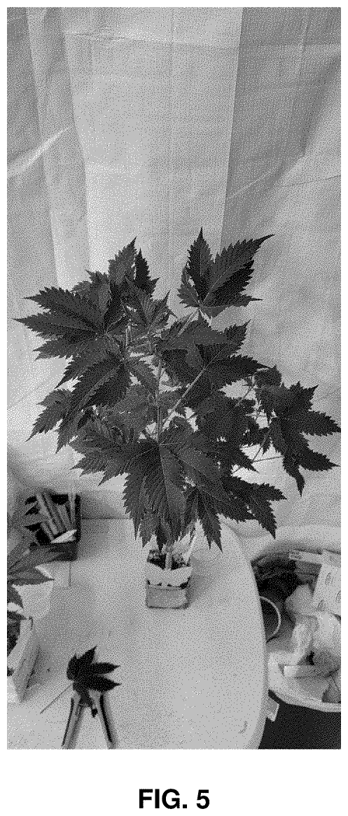

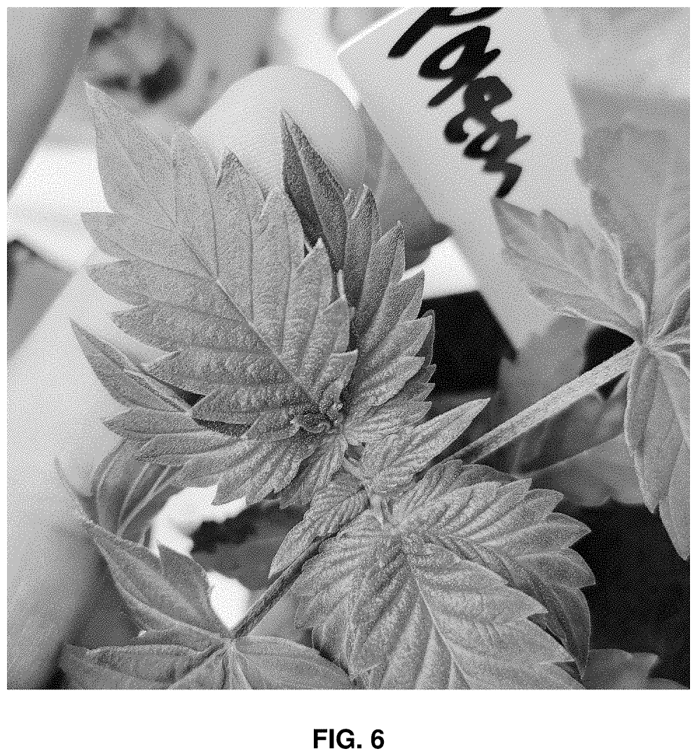

### DETAILED BOTANICAL DESCRIPTION

‘POI’ has not been observed under all possible environmental conditions, and the phenotype may vary significantly with variations in environment. The following observations, measurements, and comparisons describe this plant as grown at Mentone, Calif., when grown in the greenhouse, nursery or field, unless otherwise noted.

The color chart referenced is standard hexadecimal Web Pantone Color Chart well known to those of ordinary skill in Internet web site design.
The plant:
   Type (life form and habit).—Herbaceous tap-rooted annual.
Classification: Cultivars of Cannabis sativa.

This cultivated line possesses intoxicating properties, and so the Subspecies sativa and its varieties (var. sativa and spontanea) are eliminated from consideration.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A new and distinct cultivar of “Cannabis” plant, as shown and described.
